(19) United States
(12) Patent Application Publication
(10) Pub. No.: US 2023/0059311 A1
(43) Pub. Date: Feb. 23, 2023

(54) Bodily Soothing Assembly

(71) Applicant: Catherine Bauman, Prescott Valley, AZ (US)

(72) Inventor: Catherine Bauman, Prescott Valley, AZ (US)

(21) Appl. No.: 17/405,542

(22) Filed: Aug. 18, 2021

(51) Int. Cl. A61M 21/02; A61H 15/00

(52) U.S. Cl. CPC A61M 21/02; A61H 15/00; A61M 2021/0022

(57) ABSTRACT

A bodily soothing assembly for gently stroking a user includes a base that is positionable on a support surface. A sleeve is coupled to and extends upwardly from the base. A line extends outwardly from the sleeve and the line is comprised of a resilient material thereby facilitating the line to extend downwardly from the elbow in an arc. A massager is coupled to the line such that the massager is suspended from the sleeve. In this way the massager can rest on a user that is lying near the base. A rotation unit is integrated into the base and the rotation unit twists the line when the rotation unit is turned on thereby facilitating the massager to be rotated. In this way the massager can gently massage the user for soothing the user.

### CROSS-REFERENCE TO RELATED APPLICATIONS

Not Applicable

### STATEMENT REGARDING FEDERALLY SPONSORED RESEARCH OR DEVELOPMENT

Not Applicable

### THE NAMES OF THE PARTIES TO A JOINT RESEARCH AGREEMENT

Not Applicable

### (e) INCORPORATION-BY-REFERENCE OF MATERIAL SUBMITTED ON A COMPACT DISC OR AS A TEXT FILE VIA THE OFFICE ELECTRONIC FILING SYSTEM

Not Applicable

### STATEMENT REGARDING PRIOR DISCLOSURES BY THE INVENTOR OR JOINT INVENTOR

Not Applicable

### BACKGROUND OF THE INVENTION

### (1) Field of the Invention

The disclosure relates to soothing devices and more particularly pertains to a new soothing device for gently stroking a user. The device includes a telescopic sleeve and a resilient line that extends outwardly from the sleeve. A massage unit is attached to the line and a rotation unit in attached to the line for spinning the line. In this way the massage unit is rotated to gently stroke the user for soothing the user.

### (2) Description of Related Art Including Information Disclosed Under 37 CFR 1.97 and 1.98

The prior art relates to soothing devices including a variety of massaging machines that include a pivotable armature and a massaging unit that is suspended from the pivotable armature. The prior art discloses a massage unit that includes a plurality of flexible arms and a plurality of balls, each coupled to a respective flexible arm for massaging a user. The prior art discloses an ornamental design of a massager that includes a plurality of flexible fingers each radiating outwardly from a ball.

### BRIEF SUMMARY OF THE INVENTION

An embodiment of the disclosure meets the needs presented above by generally comprising a base that is positionable on a support surface. A sleeve is coupled to and extends upwardly from the base. A line extends outwardly from the sleeve and the line is comprised of a resilient material thereby facilitating the line to extend downwardly from the elbow in an arc. A massager is coupled to the line such that the massager is suspended from the sleeve. In this way the massager can rest on a user that is lying near the base. A rotation unit is integrated into the base and the rotation unit twists the line when the rotation unit is turned on thereby facilitating the massager to be rotated. In this way the massager can gently massage the user for soothing the user.

There has thus been outlined, rather broadly, the more important features of the disclosure in order that the detailed description thereof that follows may be better understood, and in order that the present contribution to the art may be better appreciated. There are additional features of the disclosure that will be described hereinafter and which will form the subject matter of the claims appended hereto.

The objects of the disclosure, along with the various features of novelty which characterize the disclosure, are pointed out with particularity in the claims annexed to and forming a part of this disclosure.

### BRIEF DESCRIPTION OF SEVERAL VIEWS OF THE DRAWING(S)

The disclosure will be better understood and objects other than those set forth above will become apparent when consideration is given to the following detailed description thereof. Such description makes reference to the annexed drawings wherein:

FIG. 1 is a perspective view of a bodily soothing assembly according to an embodiment of the disclosure.

FIG. 2 is a right side phantom view of an embodiment of the disclosure.

FIG. 3 is a cross sectional view taken along line 3-3 of FIG. 1 of an embodiment of the disclosure.

FIG. 4 is a top view of an embodiment of the disclosure.

FIG. 5 is a perspective view of a plurality of massage units of an embodiment of the disclosure.

FIG. 6 is a perspective in-use view of an embodiment of the disclosure.

### DETAILED DESCRIPTION OF THE INVENTION

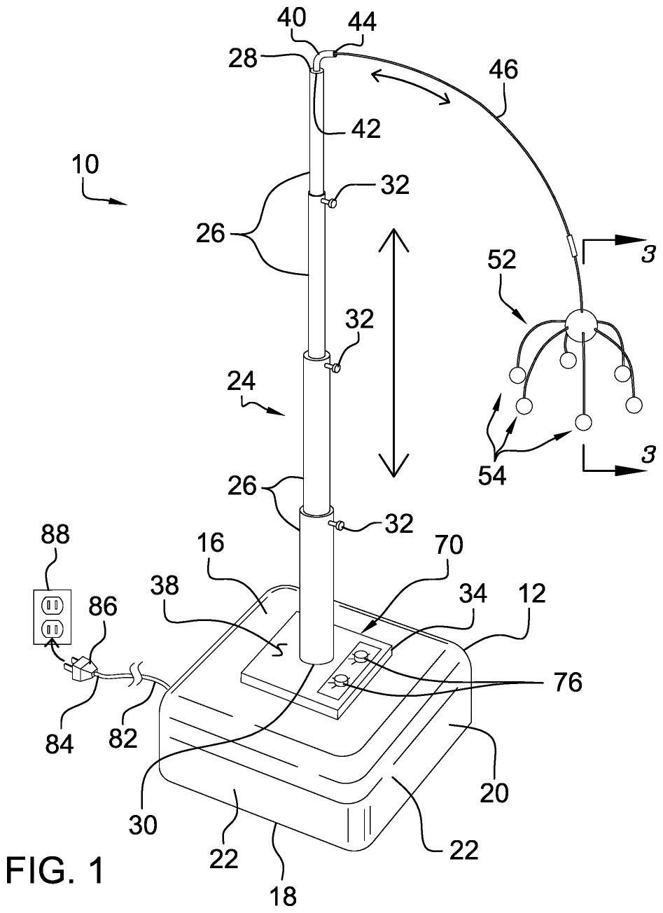

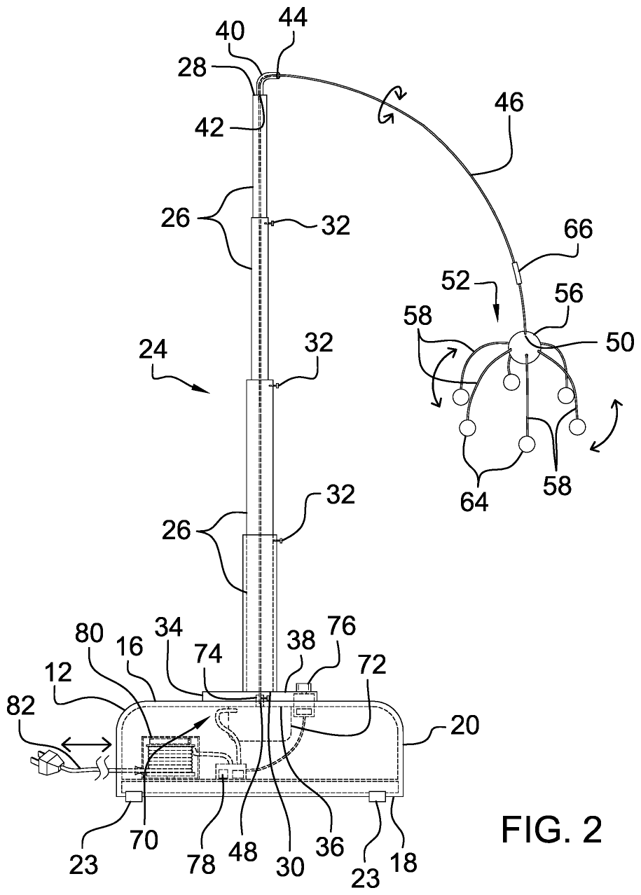

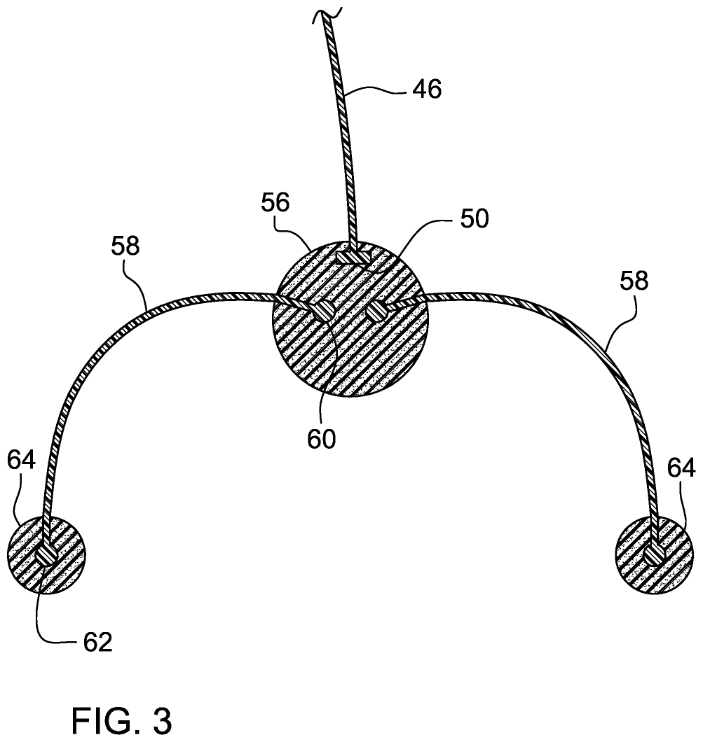

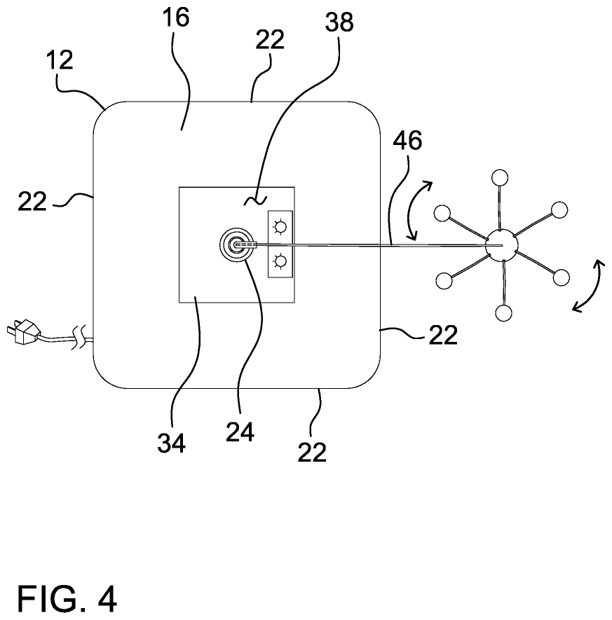

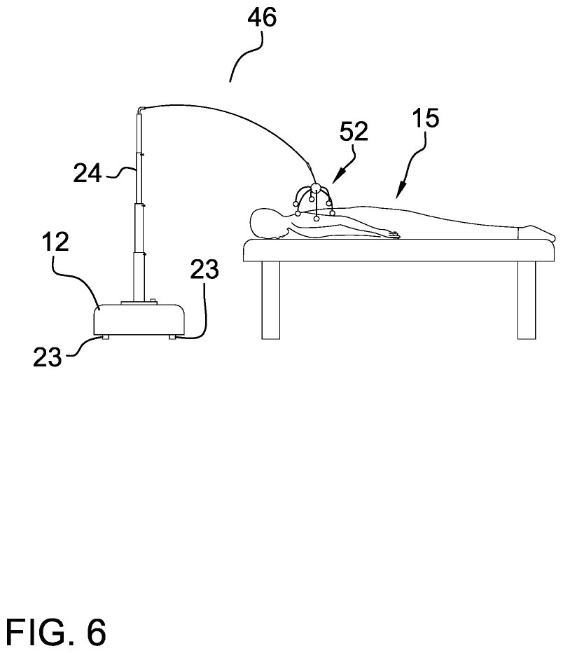

With reference now to the drawings, and in particular to FIGS. 1 through 6 thereof, a new soothing device embodying the principles and concepts of an embodiment of the disclosure and generally designated by the reference numeral 10 will be described.

As best illustrated in FIGS. 1 through 6, the bodily soothing assembly 10 generally comprises a base 12 that is weighted to inhibit the base 12 from tipping over on a support surface 14. The support surface 14 may be a night stand, a table top or other horizontal support surface that is positioned near a bed or other location that a user 15 might be lying down. Additionally, the user 15 might be a child trying to fall asleep, a person that is emotionally struggling or any other person that needs to be soothed. The base 12 has a top wall 16, a bottom wall 18 and an outer wall 20 extending between the top wall 16 and the bottom wall 18, and the outer wall 20 has a plurality of intersecting sides 22 such that the base 12 has a rectangular shape. Furthermore, the bottom wall 18 is comprised of a weighted material such as steel or other similar type of highly dense material. A plurality of rollers 23 is each rotatably integrated into the bottom wall 18 of the base 12 to facilitate the base 12 to roll on a support surface, such as a floor in a room or other horizontal support surface.

A sleeve 24 is coupled to and extends upwardly from the base 12 and the sleeve 24 comprises a plurality of sections 26 that slidably engage each other such that the sleeve 24 has a telescopically adjustable height. Additionally, the sleeve 24 has a top end 28 and a bottom end 30. A plurality of set screws 32 is provided and each of the set screws 32 extends through a respective one of the sections 26 of the sleeve 24. Each of the set screws 32 engages a respective one of the sections 26 of the sleeve 24 when the set screws 32 are tightened for retaining the sleeve 24 at a desired height.

A plate 34 is provided that has a bottom surface 36 and a top surface 38, and the bottom surface 36 is attached to the top wall 16 of the base 12. The plate 34 is centrally positioned on the top wall 16 and the bottom end 30 of the sleeve 24 is coupled to the top surface 38 of the plate 34 having the sleeve 24 being centrally positioned on the top surface 38. An elbow 40 is provided that has a first end 42 and a second end 44. The elbow 40 is hollow and the first end 42 is coupled to the top end 28 of the sleeve 24 having the second end 44 being directed away from the sleeve 24.

A line 46 is provided, the line 46 extends through the sleeve 24 and the line 46 extends outwardly from the sleeve 24. The line 46 has a primary end 48 and a secondary end 50, and the line 46 extends through the plate 34 such that the primary end 48 is positioned inside the base 12. Furthermore, the line 46 extends outwardly through the second end 44 of the elbow 40 such that the secondary end 50 is exposed. The line 46 is comprised of a resilient material thereby facilitating the line 46 to extend downwardly from the elbow 40 in an arc.

A massager 52 is coupled to the line 46 such that the massager 52 is suspended from the sleeve 24. In this way the massager 52 can rest on a user 15 that is lying near the base 12. The massager 52 includes a plurality of flexible elements 54 and each of the flexible elements 54 can gently touch the user 15. In this way the massager 52 can gently rub the user's 15 back, for example, to simulate a mother's gentle touch to soothe the user 15 to sleep or to emotionally comfort the user 15.

The massager 52 comprises a first ball 56 that is coupled to the secondary end 50 of the line 46. A plurality of fingers 58 is provided and each of the fingers 58 has a first end 60 and a second end 62. Each of the first end 60 and the second end 62 of each of the fingers 58 is rounded into a ball. Additionally, the first end 60 of each of the fingers 58 is embedded within the first ball 56 having each of the fingers 58 extending outwardly from the first ball 56. Moreover, the fingers 58 are spaced apart from each other and are distributed around an equator of the first ball 56. Each of the fingers 58 is comprised of a resilient material thereby facilitating each of the fingers 58 to extend downwardly from the first ball 56 in an arc. Furthermore, each of the fingers 58 defines a respective one of the flexible elements 54 of the massager 52.

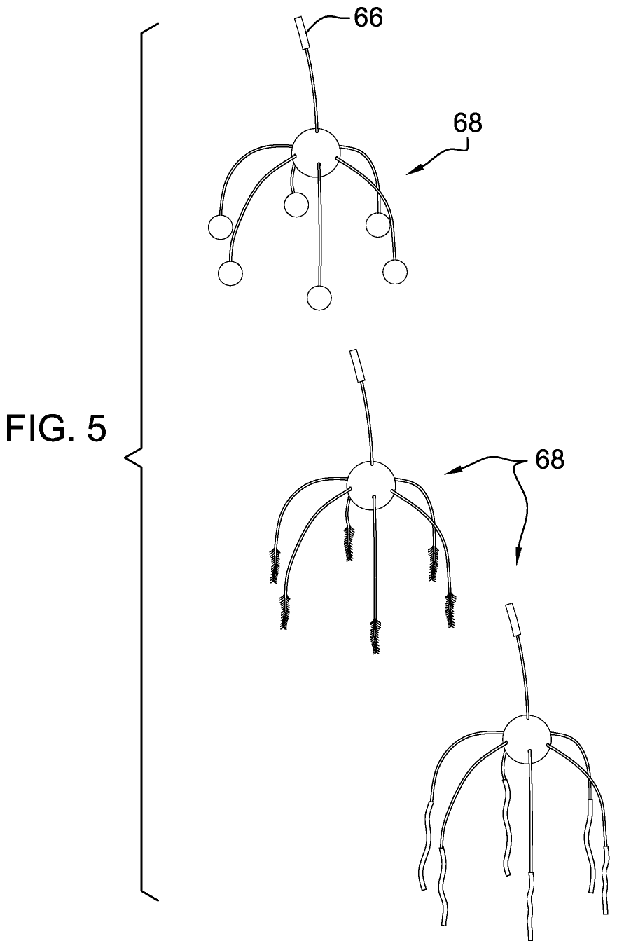

The massager 52 includes a plurality of second balls 64 and the second end 62 of each of the fingers 58 is embedded within a respective one of the second balls 64. Thus, each of the second balls 64 is suspended from the first ball 56. In this way each of the second balls 64 can rest against the user 15. Each of the second balls 64 may be comprised of a resiliently compressible material such as rubber or the like for enhancing comfort for the user 15. The line 46 may include a coupler 66 and a plurality of the massage units 68 might be provided. As is most clearly shown in FIG. 5, the flexible elements 54 on each of the plurality of massage units 68 may include brushes, feathers, ribbons or other objects that can gently touch the user 15. Additionally, the coupler 66 may facilitate the plurality of massage units 68 to be interchangeable on the line 46 to satisfy the user's 15 preference.

A rotation unit 70 is integrated into the base 12 and the line 46 is coupled to the rotation unit 70. The rotation unit 70 twists the line 46 when the rotation unit 70 is turned on thereby facilitating the massager 52 to be rotated. In this way the massager 52 can gently massage the user 15 for soothing the user 15. The rotation unit 70 comprises a motor 72 that is positioned in the base 12 and the motor 72 is positioned on the top wall 16 of the base 12. The motor 72 may comprise an electric motor or the like. The rotation unit 70 includes a receiver 74 that is coupled to the motor 72 such that the motor 72 rotates the receiver 74 when the motor 72 is turned on. The receiver 74 extends upwardly through the plate 34 and the receiver 74 insertably receives the primary end 48 of the line 46 such that the receiver 74 twists the line 46 when the motor 72 is turned on.

The rotation unit 70 includes a pair of knobs 76 and each of the knobs 76 is rotatably coupled to the plate 34 such that each of the knobs 76 is accessible to the user 15. Each of the knobs 76 is electrically coupled to the motor 72 and the pair of knobs 76 controls operational parameters of the motor 72, including but not being limited to, power on, power off and rotational speed of the motor 72. A control circuit 78 might be provided that includes a step down transformer, a bridge rectifier and other circuitry common to electric motor control circuits.

A spool 80 is provided and the spool 80 is rotatably positioned in the base 12. The spool 80 is biased to rotate in a first direction and the spool 80 is urgeable to rotate in a second direction. A power cord 82 is provided and the power cord 82 is wrapped around the spool 80. The power cord 82 extends outwardly through the outer wall 20 of the base 12 and the power cord 82 is electrically coupled to the motor 72. The power cord 82 has a distal end 84 with respect to the outer wall 20, a male plug 86 is electrically coupled to the distal end 84 and the male plug 86 can be plugged into a female electrical outlet 88. The spool 80 rotates in the second direction when the power cord 82 is pulled outwardly from the base 12 and the spool 80 rotates in the first direction when the power cord 82 is not plugged in.

In use, the base 12 is positioned in a location next to the user 15 and the sleeve 24 is adjusted to an appropriate height to facilitate each of the second balls 64 to rest on the user 15. The rotation unit 70 is turned on and the rotation unit 70 twists the line 46. In this way the first ball 56 is rotated such that each of the second balls 64 is moved in a circle. Thus, the second balls 64 gently massage the user 15 to soothe the user 15 and simulate a mother's gentle touch. Additionally, a respective one of the plurality of massage units 68 can be attached to the line 46 to satisfy the user's 15 preference.

With respect to the above description then, it is to be realized that the optimum dimensional relationships for the parts of an embodiment enabled by the disclosure, to include variations in size, materials, shape, form, function and manner of operation, assembly and use, are deemed readily apparent and obvious to one skilled in the art, and all equivalent relationships to those illustrated in the drawings and described in the specification are intended to be encompassed by an embodiment of the disclosure.

Therefore, the foregoing is considered as illustrative only of the principles of the disclosure. Further, since numerous modifications and changes will readily occur to those skilled in the art, it is not desired to limit the disclosure to the exact construction and operation shown and described, and accordingly, all suitable modifications and equivalents may be resorted to, falling within the scope of the disclosure. In this patent document, the word “comprising” is used in its non-limiting sense to mean that items following the word are included, but items not specifically mentioned are not excluded. A reference to an element by the indefinite article “a” does not exclude the possibility that more than one of the element is present, unless the context clearly requires that there be only one of the elements.

I claim:

1. A bodily soothing assembly for gently stroking a person to soothe the person, said assembly comprising:
a base being weighted wherein said base is configured to be inhibited from tipping over on a support surface;
a sleeve being coupled to and extending upwardly from said base, said sleeve comprising a plurality of sections slidably engaging each other such that said sleeve has a telescopically adjustable height;
a line extending through said sleeve, said line extending outwardly from said sleeve, said line being comprised of a resilient material thereby facilitating said line to extend downwardly from said elbow in an arc;
a massager being coupled to said line such that said massager is suspended from said sleeve wherein said massager is configured to rest on a user that is lying near said base, said massager including a plurality of flexible elements wherein each of said flexible element is configured to gently touch the user; and
a rotation unit being integrated into said base, said line being coupled to said rotation unit, said rotation unit twisting said line when said rotation unit is turned on thereby facilitating said massager to be rotated wherein said massager is configured to gently massage the user for soothing the user.

2. The assembly according to claim 1, wherein:
said base has a top wall, a bottom wall and an outer wall extending between said top wall and said bottom wall, said outer wall having a plurality of intersecting sides such that said base has a rectangular shape, said bottom wall being comprised of a weighted material;
said sleeve has a top end and a bottom end; and
said assembly includes a plate having a bottom surface and a top surface, said bottom surface being attached to said top wall of said base having said plate being centrally positioned on said top wall, said bottom end of said sleeve being coupled to said top surface of said plate having said sleeve being centrally positioned on said top surface.

3. The assembly according to claim 2, wherein:
said assembly includes a plurality of set screws, each of said set screws extending through a respective one of said sections of said sleeve, each of said set screws engaging a respective one of said sections of said sleeve when said set screws are tightened for retaining said sleeve at a desired height;
said assembly includes an elbow having a first end and a second end, said elbow being hollow, said first end being coupled to said top end of said sleeve having said second end being directed away from said sleeve; and
said line has a primary end and a secondary end, said line extending through said plate such that said primary end is positioned inside said base, said line extending outwardly through said second end of said elbow such that said secondary end is exposed.

4. The assembly according to claim 1, wherein:
said line has a primary end and a secondary end; and
said massager comprises:
a first ball being coupled to said secondary end of said line;
a plurality of fingers, each of said fingers having a first end and a second end, each of said first end and said second end of each of said fingers being rounded into a ball, said first end of each of said fingers being embedded within said first ball having each of said fingers extending outwardly from said first ball, said fingers being spaced apart from each other and being distributed around an equator of said first ball, each of said fingers being comprised of a resilient material thereby facilitating each of said fingers to extend downwardly from said first ball in an arc, each of said fingers defining a respective one of said flexible elements of said massager; and
a plurality of second balls, said second end of each of said fingers being embedded within a respective one of said second balls such that each of said second balls is suspended from said first ball wherein each of said second balls is configured to rest against the user.

5. The assembly according to claim 2, wherein said rotation unit comprises:
a motor being positioned in said base, said motor being positioned on said top wall of said base;
a receiver being coupled to said motor such that said motor rotates said receiver when said motor is turned on, said receiver extending upwardly through said plate, said receiver insertably receiving said primary end of said line such that said receiver twists said line when said motor is turned on; and
a pair of knobs, each of said knobs being rotatably coupled to said plate wherein each of said knobs is configured to be accessible to the user, each of said knobs being electrically coupled to said motor, said pair of knobs controlling operational parameters of said motor, including power on, power off and rotational speed of said motor.

6. The assembly according to claim 5, further comprising:
a spool being rotatably positioned in said base, said spool being biased to rotate in a first direction, said spool being urgeable to rotate in a second direction; and
a power cord being wrapped around said spool, said power cord extending outwardly through an outer wall of said base, said power cord being electrically coupled to said motor, said power cord having a distal end with respect to said outer wall, said distal end having a male plug being electrically coupled thereto, said spool rotating in said second direction when said power cord is pulled outwardly from said base wherein said power cord is configured to be plugged into a female electrical outlet, said spool rotating in said first direction when said power cord is not plugged in.

7. A bodily soothing assembly for gently stroking a person to soothe the person, said assembly comprising:
a base being weighted wherein said base is configured to be inhibited from tipping over on a support surface, said base having a top wall, a bottom wall and an outer wall extending between said top wall and said bottom wall, said outer wall having a plurality of intersecting sides such that said base has a rectangular shape, said bottom wall being comprised of a weighted material;
a plurality of rollers, each of said rollers being rotatably integrated into said bottom wall of said base wherein said plurality of rollers is configured to facilitate said base to roll along a support surface;
a sleeve being coupled to and extending upwardly from said base, said sleeve comprising a plurality of sections slidably engaging each other such that said sleeve has a telescopically adjustable height, said sleeve having a top end and a bottom end;
a plurality of set screws, each of said set screws extending through a respective one of said sections of said sleeve, each of said set screws engaging a respective one of said sections of said sleeve when said set screws are tightened for retaining said sleeve at a desired height;
a plate having a bottom surface and a top surface, said bottom surface being attached to said top wall of said base having said plate being centrally positioned on said top wall, said bottom end of said sleeve being coupled to said top surface of said plate having said sleeve being centrally positioned on said top surface;
an elbow having a first end and a second end, said elbow being hollow, said first end being coupled to said top end of said sleeve having said second end being directed away from said sleeve;
a line extending through said sleeve, said line extending outwardly from said sleeve, said line having a primary end and a secondary end, said line extending through said plate such that said primary end is positioned inside said base, said line extending outwardly through said second end of said elbow such that said secondary end is exposed, said line being comprised of a resilient material thereby facilitating said line to extend downwardly from said elbow in an arc;
a massager being coupled to said line such that said massager is suspended from said sleeve wherein said massager is configured to rest on a user that is lying near said base, said massager including a plurality of flexible elements wherein each of said flexible element is configured to gently touch the user, said massager comprising:
a first ball being coupled to said secondary end of said line;
a plurality of fingers, each of said fingers having a first end and a second end, each of said first end and said second end of each of said fingers being rounded into a ball, said first end of each of said fingers being embedded within said first ball having each of said fingers extending outwardly from said first ball, said fingers being spaced apart from each other and being distributed around an equator of said first ball, each of said fingers being comprised of a resilient material thereby facilitating each of said fingers to extend downwardly from said first ball in an arc, each of said fingers defining a respective one of said flexible elements of said massager; and
a plurality of second balls, said second end of each of said fingers being embedded within a respective one of said second balls such that each of said second balls is suspended from said first ball wherein each of said second balls is configured to rest against the user;
a rotation unit being integrated into said base, said line being coupled to said rotation unit, said rotation unit twisting said line when said rotation unit is turned on thereby facilitating said massager to be rotated wherein said massager is configured to gently massage the user for soothing the user, said rotation unit comprising:
a motor being positioned in said base, said motor being positioned on said top wall of said base;
a receiver being coupled to said motor such that said motor rotates said receiver when said motor is turned on, said receiver extending upwardly through said plate, said receiver insertably receiving said primary end of said line such that said receiver twists said line when said motor is turned on; and
a pair of knobs, each of said knobs being rotatably coupled to said plate wherein each of said knobs is configured to be accessible to the user, each of said knobs being electrically coupled to said motor, said pair of knobs controlling operational parameters of said motor, including power on, power off and rotational speed of said motor;
a spool being rotatably positioned in said base, said spool being biased to rotate in a first direction, said spool being urgeable to rotate in a second direction; and
a power cord being wrapped around said spool, said power cord extending outwardly through said outer wall of said base, said power cord being electrically coupled to said motor, said power cord having a distal end with respect to said outer wall, said distal end having a male plug being electrically coupled thereto, said spool rotating in said second direction when said power cord is pulled outwardly from said base wherein said power cord is configured to be plugged into a female electrical outlet, said spool rotating in said first direction when said power cord is not plugged in.
